SEQUENCE LISTING (8 sequences)

SEQ ID NO: 1 - DNA, 48 bp, synthetic construct
  misc_feature 1..48: note = Artificial nucleotide sequence for example
  source 1..48: mol_type = other DNA
actgactgactgactgcccccccttttttttttttttaaaaattgggg

SEQ ID NO: 2 - DNA, 42 bp, synthetic construct
  misc_feature 1..42: note = Artificial nucleotide sequence for example
  source 1..42: mol_type = other DNA
tttccctttgggaaaggtttggtttaaaggttggggtgaatt

SEQ ID NO: 3 - DNA, 23 bp, synthetic construct
  misc_feature 1..23: note = Artificial nucleotide sequence for example
  source 1..23: mol_type = other DNA
actgactgactgactgccccccc

SEQ ID NO: 4 - DNA, 25 bp, synthetic construct
  misc_feature 1..25: note = Artificial nucleotide sequence for example
  source 1..25: mol_type = other DNA
ttttttttttttttaaaaattgggg

SEQ ID NO: 5 - DNA, 18 bp, synthetic construct
  misc_feature 1..18: note = Artificial nucleotide sequence for example
  source 1..18: mol_type = other DNA
tttccctttgggaaaggt

SEQ ID NO: 6 - DNA, 24 bp, synthetic construct
  misc_feature 1..24: note = Artificial nucleotide sequence for example
  source 1..24: mol_type = other DNA
ttggtttaaaggttggggtgaatt

SEQ ID NO: 7 - DNA, 23 bp, synthetic construct
  misc_feature 1..23: note = Artificial nucleotide sequence for example
  source 1..23: mol_type = other DNA
actgactgactgactgccccccc

SEQ ID NO: 8 - DNA, 18 bp, synthetic construct
  misc_feature 1..18: note = Artificial nucleotide sequence for example
  source 1..18: mol_type = other DNA
tttttttttgggaaaggt

(19) United States
(12) Patent Application Publication
(10) Pub. No.: US 2025/0188511 A1
(43) Pub. Date: Jun. 12, 2025

(54) CONTAMINATION-FREE METAGENOMIC DNA SEQUENCING

(71) Applicant: CORNELL UNIVERSITY

(72) Inventors: Iwijn DE VLAMINCK, Ithaca, NY (US); Alexandre Pellan CHENG, Ithaca, NY (US); Omary MZAVA, Ithaca, NY (US); Adrienne CHANG, Ithaca, NY (US)

(73) Assignee: CORNELL UNIVERSITY, Ithaca, NY (US)

(21) Appl. No.: 18/686,146

(22) PCT Filed: Aug. 25, 2022
(86) PCT No.: PCT/US2022/075442

§ 371 Date: Feb. 23, 2024

Related U.S. Application Data
(60) Provisional application No. 63/237,367, filed on Aug. 26, 2021.

(51) Int. Cl. C12Q 1/6806; C12Q 1/34

(52) U.S. Cl. CPC C12Q 1/6806; C12Q 1/34

(57) ABSTRACT

Methods of differentiating sample-intrinsic nucleic acids from contaminant nucleic acids are described herein.

### CROSS REFERENCE TO RELATED APPLICATIONS

This application claims the benefit of U.S. Provisional Patent Application No. 63/237,367, filed Aug. 26, 2021, the contents of which are incorporated herein by reference in its entirety.

### STATEMENT REGARDING FEDERALLY SPONSORED RESEARCH

This invention was supported in part with government support under RO1AI146165 and 1RO1AI151059 awarded by the National Institutes of Health. The government has certain rights in this invention.

### INCORPORATION BY REFERENCE OF SEQUENCE LISTING

The sequence listing in the XML, named as 39830WO_9735_02_PC_SequenceListing.xml of 12 KB, created on Aug. 24, 2022, and submitted via Patent Center, is incorporated herein by reference.

### BACKGROUND

Sequencing experiments typically involve the acquisition of a sample of interest, the extraction of nucleic acids (DNA and/or RNA), and the preparation of the nucleic acids for sequencing. These steps, which require specialized enzymes, consumables and often human manipulation, can introduce contaminant nucleic acids, which cannot be distinguished from the nucleic acids of the original sample.

DNA contamination is in particular a significant problem for DNA sequencing assays to measure the provenance and abundance of microbial DNA in a sample. DNA sequencing followed by data analysis routines to compare DNA sequences to the known sequence of microbes is a very often used approach to identify and quantify the sources of microbial DNA in a sample. This approach is often called metagenomic sequencing (sequencing of meta-genomes, or the microbial genomic DNA sources represented in an environment or clinical sample). Metagenomic sequencing can be used, for example, to study the structure and function of microbial communities, such as the microbes that populate the human gut, or the human skin.

Another important application of metagenomic DNA sequencing is in the identification of putative pathogens in a clinical sample. These assays are very sensitive to contamination of DNA introduced during the assay, in particular when the biomass of microbial DNA in the sample is low, compared to the biomass of contaminant DNA introduced in the sample during the laboratory steps required for DNA sequencing.

Multiple solutions have been proposed to overcome the impact of DNA contamination on low biomass metagenomic sequencing. DNA contamination can be avoided to an extent by processing samples in a clean room facility, sterilizing consumables, and incorporating non-redundant dual indexing and unique molecular identifiers during library preparation. However, while these approaches minimize the influence of contaminant DNA, they do not avoid contaminant DNA present in reagents. Other approaches are based on batch-correction algorithms that identify microbial species detected in negative controls, those in low relative abundance, or those that are inversely correlated with DNA concentration. These indirect methods of identifying contaminant species, however, tend to overcorrect, eliminate sample-intrinsic species that are also common DNA contaminants, and make the incorrect assumption that sample contamination is perfectly reproducible across all samples in a batch.

### SUMMARY OF THE DISCLOSURE

The present disclosure is directed to methods of differentiating sample-intrinsic nucleic acids from contaminant nucleic acids.

In a first aspect, the present disclosure is directed to methods of differentiating sample-intrinsic nucleic acids from contaminant nucleic acids, the methods comprising:
providing a sample containing nucleic acids;
tagging the nucleic acids in or from the sample;
subjecting the nucleic acids after tagging to nucleic acid sequencing; and
differentiating sample intrinsic nucleic acids from contaminant nucleic acids based on tag and sequence analysis.

In some embodiments of the method, the sample is obtained from a host and the sequence analysis comprises aligning the nucleic acid sequences to a host genome thereby identifying sequences from the host. In some embodiments, the tag and sequence analysis comprises filtering the nucleic acid sequences into a preliminary group of tagged sequences and a preliminary group of untagged sequences. In some embodiments, the tag and sequence analysis comprises aligning the nucleic acid sequences with a database and identifying the contaminant reads. In some embodiments, the sample is obtained from a host, and the tag and sequence analysis comprises:

aligning the nucleic acid sequences to a host genome thereby identifying sequences from the host, and removing the host sequences;
separating the remaining nucleic acid sequences into a preliminary group of tagged sequences and a preliminary group of untagged sequences;
aligning the preliminary group of tagged sequences and the preliminary group of untagged sequences to a database thereby determining the species origin of the preliminary group of untagged sequences; and
identifying the tagged and untagged sequences of the same species origin as contaminant sequences.

In some embodiments, the sequences associated with contamination are removed from the reads. In some embodiments, the methods further comprise extracting the nucleic acids from the sample after the tagging and before subjecting the extracted nucleic acids to nucleic acid sequencing. In some embodiments, the method further comprises extracting the nucleic acids from the sample before the tagging. In some embodiments, the sample comprises viruses, microorganisms, plants, and/or mammalian cells, and the nucleic acids are extracted therefrom for sequencing. In some embodiments, the microorganisms are parasites, protists, archaea, bacteria, and/or fungi. In some embodiments, the mammalian cells are human, rodent, primate, equine, canine, bovine, and porcine cells. In some embodiments, the sample is a biological sample obtained from a mammalian host subject. In some embodiments, the biological sample is a cell free sample comprising DNA. In some embodiments the biological sample obtained from mammalian host subject is a nasal swab, a saliva sample, a skin sample, a urine sample, a gastrointestinal tract sample, a tissue sample, a fecal sample, a blood sample, a plasma sample, cerebrospinal fluid sample, peritoneal fluid sample, pleural effusion, and/or a serum sample. In some embodiments, the sample is an environmental sample. In some embodiments, the environmental sample is air, water, soil, biological materials, and wastes, wherein the wastes are liquids, solids or sludges. In some embodiments, the tagging comprises modifying one or more nucleotides of the nucleic acids in the sample. In some embodiments, the modifying comprises converting cytosine nucleotides into uracil nucleotides. In some embodiments, the converting cytosine nucleotides into uracil nucleotides is achieved by treating the nucleic acids with a bisulfite salt. In some embodiments, the converting cytosine nucleotides into uracil nucleotides is achieved by treating the nucleic acids with a cytosine deaminase, a cytidine deaminase (CDA), or a dCMP deaminase (DCTD). In some embodiments, the CDA is an apolipoprotein B editing complex (APOBEC) and/or an activation-induced deaminase. In some embodiments, the APOBEC is APOBEC1, APOBEC3A-H, and/or APOBEC3G. In some embodiments, the modifying comprises converting adenosine nucleotides to inosine nucleotides. In some embodiments, the converting adenosine nucleotides to inosine nucleotides is achieved by treating the nucleic acids with an adenosine deaminase. In some embodiments, the adenosine deaminase is AMP deaminase (AMPD1), adenosine deaminase 1 (ADA1), and/or adenosine deaminase 2 (ADA2). In some embodiments, the modifying comprises converting guanosine nucleotides to xanthosine nucleotides. In some embodiments, the converting guanosine nucleotides to xanthosine nucleotides is achieved by treating the nucleic acids with a guanine deaminase (GDA). In some embodiments, the methods further comprise after the nucleic acid sequencing:
identifying nucleic acids that do not have cytosines replaced with uracils as contaminant nucleic acids;
identifying nucleic acids that have cytosines replaced with uracils as intrinsic nucleic acids; and/or
identifying nucleic acids that have cytosines replaced with uracils wherein the uracils are converted to thymines in subsequent DNA synthesis steps as intrinsic nucleic acids.

In some embodiments, the tagging comprises attaching a compound to the nucleic acids. In some embodiments, the attaching is by way of a covalent linkage. In some embodiments, the compound is selected from biotin, a peptide, an alkyne, an azide, a BrdU, an IrdU, an amine, or an oligonucleotide. In some embodiments, the peptide is a HIS tag. In some embodiments, the oligonucleotide is a primer sequence. In some embodiments, the methods further comprise isolating the nucleic acids attached with the compound before subjecting the isolated nucleic acids to nucleic acid sequencing. In some embodiments, the extracting the nucleic acids comprises lysing the cells in the sample to release the nucleic acids. In some embodiments, the sample is suspected to contain a pathogenic microorganism. In some embodiments, the nucleic acid sequencing is metagenomic sequencing. In some embodiments, the methods further comprise enriching the non-contaminant nucleic acids by their tags.

### BRIEF DESCRIPTION OF THE DRAWINGS

The patent or application file contains at least one drawing executed in color. Copies of this paper or patent application publication with color drawing(s) will be provided by the Office upon request and payment of the necessary fee.

FIG. 1A-F. Sample-Intrinsic microbial DNA Found by Tagging and sequencing (SIFT-seq) proof-of-principle. A) Experimental workflow. Tagging of sample-intrinsic DNA by bisulfite DNA treatment is performed directly on urine or plasma. Contaminating DNA introduced after the tagging step is identified based on lack of cytosine conversion. B) Schematic of bioinformatics workflow. C) Representative example of the cytosine fraction of mapped reads in an unfiltered (top) dataset, a read-level filtered dataset (middle) and a fully filtered dataset (bottom). D) Number of reads assigned to Cutibacterium acnes (common environmental DNA contaminant) in ΦX174 DNA after conventional sequencing (green) and SIFT-seq (purple). E) Deliberate contamination assay. Detection of known contaminants before (top) and after (bottom) filtering. F) Number of reads assigned to contaminants. Boxes in the boxplots indicates 25th and 75th percentile, the band in the box indicated the median and whiskers extend to 1.5× Interquartile Range (IQR) of the hinge. Outliers (beyond 1.5×IQR) are plotted individually.

FIG. 2A-B. Comparison of traditional metagenomic DNA sequencing versus SIFT-seq metagenomic DNA sequencing. A) Standard workflow for metagenomic DNA sequencing. B) Workflow for Biotin labeling of sample intrinsic molecules. FIG. 2B, shows biotin labelling of sample-intrinsic DNA molecules in sample. After labelling, samples were subjected to normal DNA isolation workflow. Next, DNA molecules are tagmented and adapters are attached. DNA pull down step is then performed using streptavidin coated magnetic beads that bind to biotinylated DNA molecules. This filters biotinylated DNA (sample-intrinsic) and leaves out unlabeled DNA molecules (contaminants). Finally, indexes are added to sample-intrinsic DNA libraries bound to the magnetic beads then amplified, purified, and sequenced.

FIG. 3. Number of reads mapping to spiked in species. SIFT-seq assay applied to biotinylated (5 samples) and non-biotinylated (n=6) commercial phiX DNA samples at varying biomass and then spiked in 1 ng of DNA from microbial community containing DNA from 10 species (8 bacteria, 2 fungi). This figure shows the number of reads mapping to the spike in microbial community DNA. TOP ROW: PhiX DNA→Microbial community DNA standard spike-in→Library Prep→Sequencing; BOTTOM ROW: PhiX DNA→Biotinylation→Microbial community DNA standard spike in→Tagmentation→Magnetic (streptavidin) pulldown→Library amplification and clean up→Sequencing.

FIG. 4A-E. SIFT-seq applied to cell-free DNA in urine and plasma. A) Microbial abundance of 25 most abundant common contaminant genera (selected from the 68 genera4) before and after SIFT-seq filtering in plasma and urine from six independent subject cohorts (Tx=transplant). Total abundance of all contaminant genera B) and C. acnes C) before and after SIFT-seq filtering (KUCP=Kidney Transplant cohort with positive urine culture, KUCN=Kidney Transplant cohort with negative urine culture, EPTx=Early Post Transplant cohort). Bray-Curtis dissimilarity index before D) and after E) filtering. Samples are organized by: sequencing batch, researcher performing the experiment, cohort, and biofluid. Boxes in the boxplots indicates 25th and 75th percentile, the band in the box indicated the median and whiskers extend to 1.5× Interquartile Range (IQR) of the hinge. Outliers (beyond 1.5×IQR) are plotted individually. *** p-value<0.001.

FIG. 5A-D. Application of SIFT-seq to urine. A) Heatmap of abundance of species (molecules per million, MPM, species with at least one read detected by BLAST) identified in patients with and without urine culture-confirmed UTIs, before and after application of SIFT-seq filter (black * indicates agreement with urine culture). B) Boxplot of the relative number of microbe-derived molecules (MPM) in samples from patients with and without urine culture-confirmed UTIs, before and after SIFT-seq filtering. C) Sample collection timepoints after transplantation for 5 patients. D) Boxplot showing Bray-Curtis similarity index (as defined in C) of the urine microbiome within individual patients and between patients before and after stent removal. Boxes in the boxplots indicates 25th and 75th percentile, the band in the box indicated the median and whiskers extend to 1.5× Interquartile Range (IQR) of the hinge. Outliers (beyond 1.5×IQR) are plotted individually. (* p-value<0.05, ** p-value<0.01, *** p-value<0.001).

FIG. 6. Boxplot showing Bray-Curtis similarity index of the urine microbiome between patients and within individual patients before and after stent removal for the unfiltered datasets (Bray-Curtis Similarity 0.55±0.11 and 0.67±0.2 respectively). Boxes in the boxplots indicates 25th and 75th percentile, the band in the box indicated the median and whiskers extend to 1.5× Interquartile Range (IQR) of the hinge.

FIG. 7A-B. Benchmarking SIFT-seq against Low Biomass Background Correction (LBBC). A) Boxplot of the total abundance (molecules per million, MPM) of contaminant genera before and after SIFT-seq or LBBC filtering (two tailed, Wilcoxon test, PSIFT-seq <0.001, PLBBC<0.001). B) Heatmap of abundance of species (MPM) identified in patients with and without UTI, before and after application of LBBC filter. (Red * indicated species that were identified by culture and standard sequencing but removed after LBBC filtering). Boxes in the boxplots indicates 25th and 75th percentile, the band in the box indicated the median and whiskers extend to 1.5× Interquartile Range (IQR) of the hinge. Outliers (beyond 1.5×IQR) are plotted individually. *** p-value<0.001

FIG. 8. Application of SIFT-seq filtering on negative, blank control samples.

FIG. 9A-G. Application of SIFT-seq to plasma. Heatmaps of the abundance of species identified in plasma from COVID-19 patients with and without culture confirmed A) lung and B) blood infection, before and after application of SIFT-seq filter (black * indicates agreement with culture; red * indicates detection by sputum culture only; HCMV: Human cytomegalovirus, HSV-1: Herpes simplex virus 1). C) A heatmap of abundance of species identified in the sepsis cohort before and after SIFT-seq filtering (black * indicates species identified by blood culture). D) Barplot of the prevalence of Epstein-Barr Virus (EBV), Torque teno virus (TTV), malaria-causing, or shigellosis-causing microorganisms in different patient cohorts. E) Heatmap of the abundance of species identified in matched stool and plasma cfDNA samples in patients diagnosed with Crohn's disease or ulcerative colitis. F) Schematic for matched stool and plasma samples from individuals before and after medical therapy. G) Heatmap of the change in abundance of gut specific bacteria before and after treatment.

FIG. 10. Barplot of the prevalence of Epstein-Barr Virus (EBV), Torque teno virus (TTV), Malaria, or Shigellosis microorganisms in different patient cohorts before SIFT-seq filtering.

### DETAILED DESCRIPTION

The current disclosure is directed to methodologies to mitigate the problem of nucleic acid contamination in nucleic acid sequencing experiments. The methodologies involve tagging nucleic acids intrinsic to the biological sample, such that proper identification of the tag combined with sequence analysis can be used to distinguish nucleic acids of the sample from contaminant nucleic acids.

As used herein, the term “nucleic acid” has its general meaning in the art and refers to refers to a coding or non-coding nucleic sequence. Nucleic acids include DNA (deoxyribonucleic acid) and RNA (ribonucleic acid) nucleic acids. Examples of nucleic acid thus include but are not limited to DNA, mRNA, tRNA, IRNA, tmRNA, miRNA, piRNA, snoRNA, and snRNA. Nucleic acids thus encompass coding and non-coding region of a genome (i.e., nuclear or mitochondrial).

The present disclosure is unique in the sense that it provides a physical approach to deal with the problem of environmental contamination in metagenomic DNA sequencing.

The methods disclosed herein are direct, inference-free methods to remove contaminants in that it relies on the sequencing of the actual sample, without requiring batches (to measure other sample biomasses) and controls (to identify contaminant species). Contaminants can be physically removed, or computationally removed directly by identifying the sample-specific tag. Direct measurements are particularly important in clinical settings, as samples do not need to be processed with other samples or controls.

This disclosure provides alternative methods to tag and remove contaminant nucleic acid molecules. One aspect of the disclosure introduces base pair mutations into the sample-intrinsic nucleic acid molecules. The introduction of known mutations into true molecules can be used to remove contaminant molecules that lack this mutation. For example, conversion of all or substantially all cytosines to thymines (using bisulfite salt) can be used prior to nucleic acid extraction. After sequencing, nucleic acid molecules that are C-rich (i.e., unconverted) can be removed physically or computationally as contaminant nucleic acids.

Another aspect of the disclosure labels the sample-intrinsic nucleic acid molecules. Labelling true molecules with a physical tag, can be done prior to and during nucleic acid extraction. After library preparation, select chemical affinities can be leveraged to attract tagged molecules and exclude non-tagged molecules. Sequencing of these molecules can then be performed.

As used herein, “tagged” nucleic acids can refer to nucleic acid molecules onto which a physical tag is placed. Methods of tagging or labelling nucleic acids with a physical marker are known in the art. Examples of such physical tagging are radioactive labels, biotin, dinitrophenyl, and fluorescent tags. In some embodiments, the tagging comprises attaching a compound to the nucleic acids. This compound can be attached by way of a covalent linkage. In some embodiments, the compound is selected from biotin, a peptide, an alkyne, an azide, a BrdU, an IrdU, an amine, or an oligonucleotide. In some embodiments, the peptide is a HIS tag. In some embodiments, the oligonucleotide is a primer sequence.

Additionally, “tagged” nucleic acids can refer to nucleic acids in which one or more nucleotides has been knowingly modified for purposes of the present methods. For example, conversion of all or substantially all cytosines to thymines/uracils can be used and considered as one way of tagging. After sequencing, nucleic acid molecules that are C-rich (i.e., unconverted and thus untagged) can be removed. Modifications which introduce nucleic acid mutations are known in the art. Examples of modifications that can be used for the disclosed methods include, but are not limited to, conversion of cytosine to thymine/uracil through bisulfite salt or through enzymes such as cytosine deaminase, a cytidine deaminase (CDA), or a dCMP deaminase (DCTD) including the AID/APOBEC deaminases. Such AID/APOBEC deaminases include APOBEC1, APOBEC3A-H, and/or APOBEC3G. Additional manipulation can occur by converting adenosine to inosine through the use of an adenosine deaminase including AMP deaminase (AMPD1), adenosine deaminase 1 (ADA1), and/or adenosine deaminase 2 (ADA2). Other methods of nucleotide manipulation include converting guanosine nucleotides to xanthosine nucleotides by treating the nucleic acids with a guanine deaminase (GDA).

In some embodiments, tagging comprises modifying one or more nucleotides of the nucleotides in the sample.

In some embodiments, the modifying comprises converting cytosines into uracils. In some embodiments, the converting is achieved by treating the nucleic acids with a bisulfite salt. In some embodiments, the converting is achieved by treating the nucleic acids with a cytosine deaminase, acytidine deaminase (CDA), or dCMP deaminase (DCTD). Non-limiting examples of CDAs include Apolipoprotein B mRNA editing enzyme, catalytic polypeptide 1 (APOBEC1), Apolipoprotein B mRNA editing enzyme, catalytic polypeptide-like 3A-3H (APOBEC3A-H), Apolipoprotein B mRNA editing enzyme, catalytic polypeptide-like (APOBEC3G), and activation-induced cytidine deaminase (AICDA).

In some embodiments, the modifying comprises converting adenosines to inosines. In some embodiments, the converting is achieved by treating the nucleic acids with an adenosine deaminase. Non-limiting examples of adenosine deaminases include AMP deaminase (AMPD1), adenosine deaminase 1 (ADA1) or adenosine deaminase 2 (ADA2).

In some embodiments, the modifying comprises converting guanosines to xanthosines. In some embodiments, the converting is achieved by using a guanine deaminase (GDA).

In some embodiments, the method further comprises, after the nucleic acid sequencing, identifying nucleic acids that do not have cytosines replaced with uracils as contaminant nucleic acids. In some embodiments, nucleic acids that are C-rich are identified as contaminant nucleic acids. In some embodiments, nucleic acids that contain at least 1, 2, 3, 4 or more cytosines that are not replaced with uracils per read are considered as being C-rich. In some embodiments, the reads are about 60-90 bp in length. In some embodiments, the reads are about 65-85 bp in length. In some embodiments, the reads are about 70-80 bp in length. In some embodiments, the reads are about 75 bp in length. In some embodiments, nucleic acids that contain at least about 1% or more cytosines per read are considered C-rich. For example, having 1 cytosine over a read of 75 bp would constitute 1.3% cytosine for this read. In some embodiments, nucleic acids that contain at least about 1.1% or more cytosines per read are considered C-rich nucleic acids. In some embodiments, nucleic acids that contain at least about 1.2% or more cytosines per read are considered C-rich nucleic acids. In some embodiments, nucleic acids that contain at least about 1.3% or more cytosines per read are considered C-rich nucleic acids. In some embodiments, nucleic acids that contain at least about 1.4% or more cytosines per read are considered C-rich nucleic acids. In some embodiments, nucleic acids that contain at least about 1.5% or more cytosines per read are considered C-rich nucleic acids. In some embodiments, nucleic acids that contain at least about 2% or more cytosines per read are considered C-rich nucleic acids. In some embodiments, nucleic acids that contain at least about 3% or more cytosines per read are considered C-rich nucleic acids. In some embodiments, nucleic acids that contain at least about 4% or more cytosines per read are considered C-rich nucleic acids. In some embodiments, nucleic acids that contain at least about 5% or more cytosines per read are considered C-rich nucleic acids. In some embodiments, nucleic acids that are not considered C-rich are identified as contaminant nucleic acids on the basis that such nucleic acids have been determined as being from the same species as a contaminant nucleic acid (e.g., a C-rich nucleic acid).

In some embodiments, the tagging comprises attaching a compound to the nucleic acids. In some embodiments, the tagging comprises covalently linking a compound to the nucleic acids.

In some embodiments, the compound is selected from biotin, a peptide (e.g., a HIS tag), an alkyne, an azide, a BrdU, an IrdU, an amine, or an oligonucleotide (e.g., a primer sequence). In some embodiments, alkynes can be purified via azide magnetic beads through click chemistry reactions, azides can be purified via alkyne magnetic beads or DBCO magnetic beads through click chemistry reactions, BrdUTP/IrdU can be purified indirectly with appropriate antibodies, e.g. anti-BrdUTP, primer sequence can enable enrichment in subsequent steps, and amines can be purified via carboxylate-modified or amine-blocked magnetic beads.

The methodologies disclosed herein are particularly useful for sequencing projects, including general metagenomic sequencing, low-biomass metagenomic sequencing, pathogen detection and infectious disease diagnosis.

In some embodiments, the disclosure is directed to a method of differentiating sample-intrinsic nucleic acids and contaminant nucleic acids, the method comprising: providing a sample containing nucleic acids; tagging the nucleic acids in the sample; extracting the nucleic acids from the sample; subjecting the extracted nucleic acids to nucleic acid sequencing; and identifying nucleic acids that do not contain the tag as contaminant nucleic acids.

In some embodiments, the tagging is performed immediately after the sample is collected. In some embodiments, the tagging is performed before extracting the nucleic acids from the sample. In some embodiments, the tagging is performed during nucleic acid extraction. In some embodiments, the tagging is performed after extracting the nucleic acids from the sample but before subjecting the extracted nucleic acids to nucleic acid sequencing.

In some embodiments, the sample is obtained from a host and the sample analysis comprises aligning the nucleic acid sequences obtained from sequencing to a host genome, thereby allowing identification of sequences from the host. A host genome, as used herein, refers to the genome of the where the sample was attained. For example, if the sample is a urine sample from a human, the host genome would be the human genome.

In some embodiments, the tag and sequence analysis comprises aligning the nucleic acid sequences with a database and identifying the contaminant reads. In some embodiments, the tag and sequence analysis comprises filtering the nucleic acid sequences into a preliminary group of tagged sequences and a preliminary group of untagged sequences (e.g., C-rich sequences). Tagged sequences refer to sequences of tagged nucleic acids (e.g., sequences have no or few Cs), while untagged sequences refer to sequences of nucleic acids not tagged. The term “preliminary” indicates that the determination that a sequence is tagged or untagged is preliminary because, for example, a sequence which does not have cytosines or may have only a few cytosine could be a result that the cytosines present in the original sequence were manipulated to uracil, or it could be because the original sequence does not contain cytosine.

In some embodiments, the sample is obtained from a host and the tag and sequence analysis comprises wherein the sample is obtained from a host, and the tag and sequence analysis comprises aligning the nucleic acid sequences to a host genome thereby identifying sequences from the host, and removing the host sequences; separating the remaining nucleic acid sequences into a preliminary group of tagged sequences and a preliminary group of untagged sequences; aligning the preliminary group of tagged sequences and the preliminary group of untagged sequences to a database thereby determining the species origin of the preliminary group of untagged sequences, and identifying the tagged and untagged sequences of the same species origin as contaminant sequences.

Another aspect of the disclosure is directed to a method of differentiating sample-intrinsic nucleic acids and contaminant nucleic acids, the method comprising: providing a sample containing nucleic acids; extracting the nucleic acids from the sample; tagging the nucleic acids in the sample; subjecting the extracted nucleic acids to nucleic acid sequencing; and identifying nucleic acids that do not contain the tag as contaminant nucleic acids, wherein the tagging is performed after extracting the nucleic acids from the sample but before subjecting the extracted nucleic acids to nucleic acid sequencing.

In some embodiments, the sample comprises viruses, microorganisms, plants, and/or mammalian cells, and the nucleic acids are extracted therefrom for sequencing. In some embodiments, the microorganisms can be parasites, protists, archaea, bacteria, and/or fungi. In some embodiments, the mammalian cells are human, rodent, primate, equine, canine, bovine, and porcine cells.

In some embodiments, the sample is a biological sample obtained from a mammalian host subject. In some embodiments, the mammalian subject is a human. In some embodiments, the biological sample is a cell free sample comprising nucleotides. In some embodiments, the biological sample is as a nasal swab, a saliva sample, a skin sample, a urine sample, a gastrointestinal tract sample, a tissue sample, a fecal sample, a blood sample, a plasma sample, cerebrospinal fluid sample, peritoneal fluid sample, pleural effusion, and/or a serum sample.

In some embodiments, the sample is an environmental sample. In some embodiments, the environmental sample is a water sample. In some embodiments, the environmental sample is a soil sample. In some embodiments, the environmental sample is air, biological materials, or wastes, wherein the wastes are liquids, solids, or sludges.

In some embodiments, the method further comprises, after extracting the nucleic acids from the sample, isolating the nucleic acids attached with the compound, and subject the isolated nucleic acids to nucleic acid sequencing.

In some embodiments, the extracting comprises lysis of the cells in the sample to release the nucleic acids. Methods for cell lysis vary, as recognized by those skilled in the art, depending on the sample volume and sample composition. Non-limiting methods for cell lysis include mechanical disruption, liquid homogenization, sonication, freeze-thaw, manual grinding, chemical disruption, and enzyme treatment.

In some embodiments, the sample is suspected to contain a pathogenic microorganism. In some embodiments, the pathogenic microorganism is a bacteria. In some embodiments, the pathogenic microorganism is a virus. In some embodiments, the pathogenic microorganism is a fungus.

In some embodiments, the nucleic acid sequencing is metagenomic sequencing. Methods for metagenomic sequencing vary, as recognized by those skilled in the art, and include but are not limited to Illumina sequencing and Nanopore sequencing.

In some embodiments, the method further comprises enriching the non-contaminant nucleic acids by their tags. As used herein, “enriching” is the process in which genomic regions are selectively captured from a DNA sample before sequencing. Methyl-DNA immunoprecipitation (Me-DIP) and Methyl-CpG Binding Domain (MBD)-based capture are both effective methods to enrich methylated DNA from genomic DNA samples. By additionally enriching the non-contaminant nucleic acids by the tags placed through the disclosed methods, further enrichment is possible.

Unless defined otherwise, all technical and scientific terms used herein have the same meaning as commonly understood by one skilled in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

The present methods are further supported and illustrated by the examples and experimental description.

### EXAMPLES

The following examples are presented to illustrate the present disclosure. The examples are not intended to be limiting in any manner.

### Example 1. SIFT-Seq Working Principle, Host Genome and Contaminant Genome

In a generalized example, a sample is obtained and comprises a sample-intrinsic nucleic acid. A contaminant nucleic acid is subsequently introduced into to the sample and has a sequence: ACTGACTGACTGACTGCCCCCCCTTTTTTTTTTTTTTAAAAATTGGGG (SEQ ID NO: 1). The sample-intrinsic nucleic acid has a sequence:

(SEQ ID NO: 2)
TTTCCCTTTGGGAAAGGTTTGGTTTAAAGGTTGGGGTGAATT.

For simplicity of this example, the contaminant and sample-intrinsic nucleic acids each comprise two fragments (e.g., as a result of the size limit of sequencing reads), as represented below:

Contaminant 1:
(SEQ ID NO: 3)
ACTGACTGACTGACTGCCCCCCC
Contaminant 2:
(SEQ ID NO: 4)
TTTTTTTTTTTTTTAAAAATTGGGG
SI 1:
(SEQ ID NO: 5)
TTTCCCTTTGGGAAAGGT
SI 2:
(SEQ ID NO: 6)
TTGGTTTAAAGGTTGGGGTGAATT

After bisulfite tagging and sequencing, contaminant read 1 produces: ACTGACTGACTGACTGCCCCCCC (SEQ ID NO: 7) (where the Cs are untagged) while contaminant read 2 produces: TTTTTTTTTTTTTTAAAAATTGGGG (No change from SEQ ID NO: 4). The sample-intrinsic nucleic acids are manipulated by the bisulfite treatment, changing the Cs to Ts. Therefore, SI read 1 produces: TTTTTTTTTGGGAAAGGT (SEQ ID NO: 8) (Cs are read as Ts), while SI read 2 produces: TTGGTTTAAAGGTTGGGGTGAATT (No change from SEQ ID NO: 6).

Once the reads are provided, the analytical steps are performed. The first analytical step is that of the host read removal. Here, the reads are aligned to a host genome. The host genome is the organism from which the sample is taken. For instance, a human urine sample would use the human genome as the host genome. Once the reads are aligned to a host genome, the sample-intrinsic nucleic acids align with the host genome and the remaining reads are contaminant read 1: ACTGACTGACTGACTGCCCCCCC (SEQ ID NO: 7) (Cs are untagged) and contaminant read 2: TTTTTTTTTTTTTTAAAAATTGGGGGGG (No change from SEQ ID NO: 4).

The second analytical step performed is read level filtering. This step actively looks for the tag. When using bisulfite as the chemical tagging mechanism (which transforms Cytosines into Thymines), sequences with many (or little) cytosines are looked for and placed into preliminary bins (tagged and untagged sequences). Since contaminant read 1 has many cytosines, it is placed into a preliminary group of untagged sequences, i.e., reads with sequences having many Cs. Since contaminant read 2 has few cytosines, it is place into a preliminary group of tagged sequences, i.e., reads with sequences having few Cs. The term “preliminary” indicates that the determination that the sequence is tagged is preliminary because of the possibility that the sequence before the tagging step does not include cytosine or has few cytosines.

The third analytical step performed is species level filtering. In this step, the reads are aligned to a database and identified. Read 1 is known to be a contaminant since read 1 was not tagged by the bisulfite and contains many Cs. Therefore, read 2 will be identified in this step as belonging to the same bacteria as read 1 and will be considered a contaminant even though it does not contain taggable cytosines. Therefore, both contaminant read 1 and contaminant read 2 can be computationally removed as contaminant sequences from further analysis.

### Practical Implementation of SIFT-Seq.

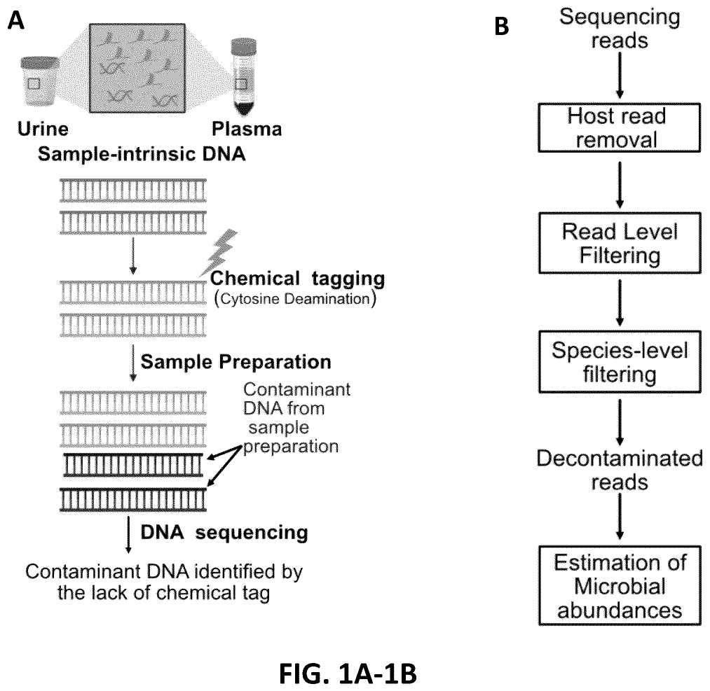

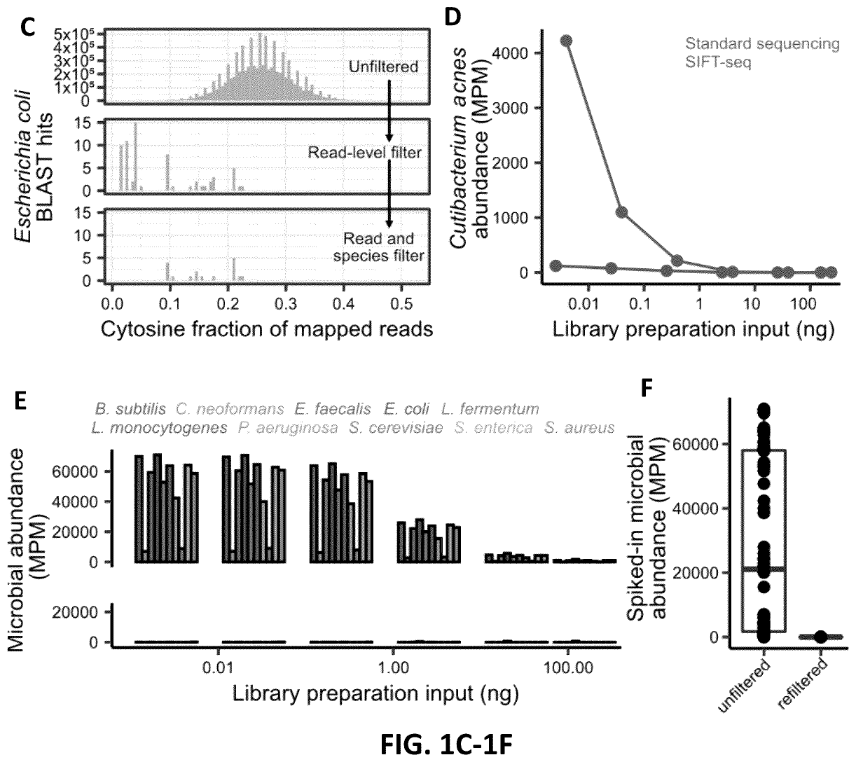

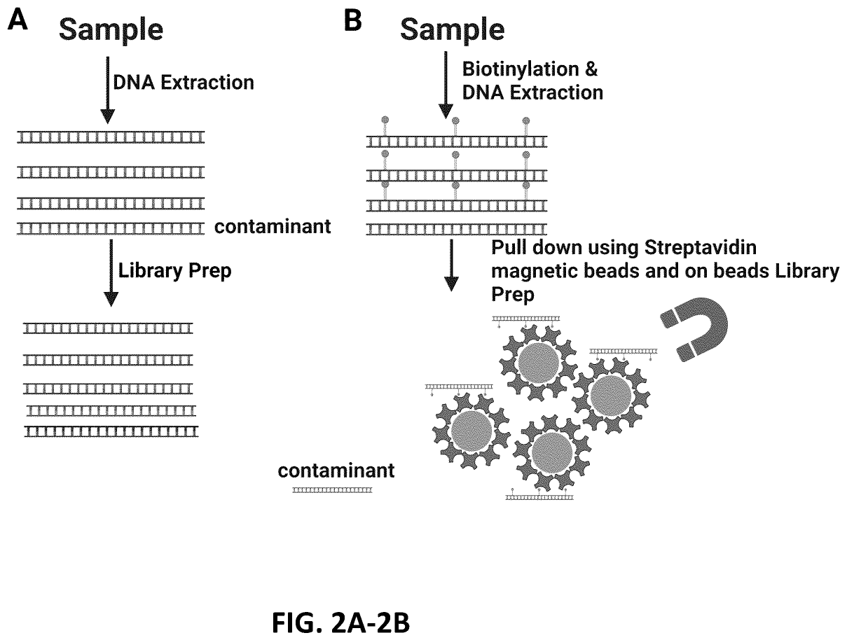

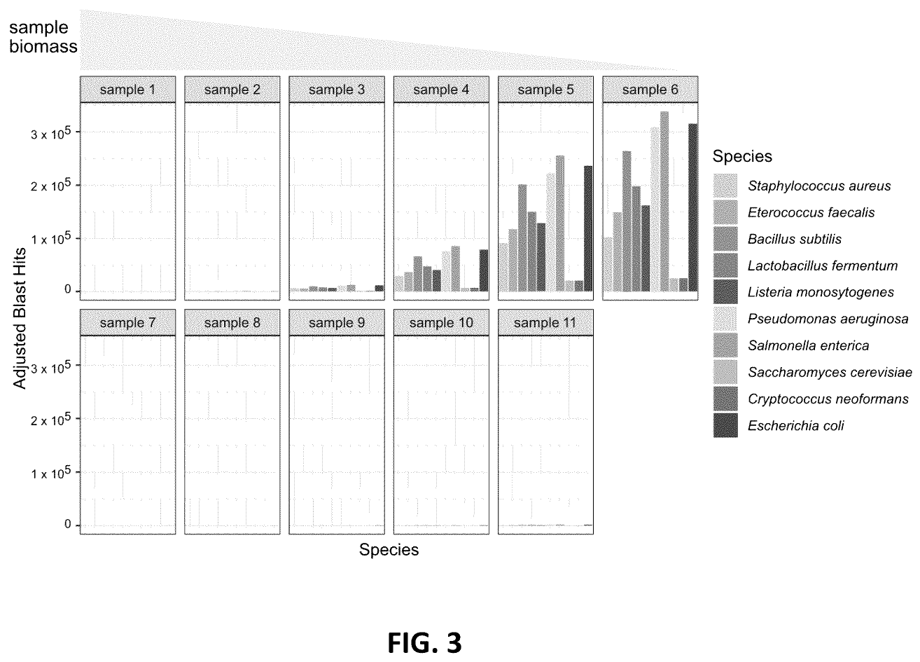

For the practical implementation of Sample-Intrinsic microbial DNA Found by Tagging and sequencing (SIFT-seq), DNA was tagged by bisulfite salt-induced conversion of unmethylated cytosines to uracils, as shown in FIG. 1A. Uracils created by bisulfite treatment are converted to thymines in subsequent DNA synthesis steps that are part of DNA sequencing library preparation. After DNA sequencing, contaminating DNA introduced after tagging can then be directly identified based on the lack of cytosine conversion. Bisulfite conversion does not require the use of commercial enzymes or oligos that are a frequent source of DNA contamination, and we found that it can be applied directly to the original sample, before DNA isolation. A bioinformatics procedure was developed to differentiate sample-intrinsic microbial DNA, contaminant microbial DNA, and host-specific DNA after SIFT-seq tagging. FIG. 1B. This procedure consists of three steps. First, host cfDNA is removed via mapping and k-mer matching. Given that CpG dinucleotides are heavily methylated in the human genome and rarely in microbial genomes, sequences containing CG dinucleotides are also removed. Second, remaining sequences that consist of more than three cytosines, or one cytosine-guanine dinucleotide are flagged and removed as likely contaminants. Last, a species-level filtering step is performed to remove any remaining reads that primarily originate from C-poor regions in the reference genome. An example of this can be seen in FIG. 1C where the cytosine fraction of mapped reads is shown in filtered and unfiltered levels.

Two assays were devised to test the principle of SIFT-seq. First, SIFT-seq and conventional DNA sequencing were applied to samples of sheared ΦX174 DNA (New England Biolabs, #N3021S) with variable biomass (0.0025 ng, 0.025 ng, 0.25 ng, 2.5 ng, 26 ng, and 155 ng for SIFT-seq; 0.004 ng, 0.04 ng, 0.4 ng, 4 ng, 35 ng, and 240 ng for standard cfDNA sequencing). The abundance of Cutibacterium acnes (C. acnes) was first quantified. C. acnes is a frequent member of the normal skin flora and is routinely identified as a contaminant in DNA sequencing. An increase in C. acnes abundance was observed with decreasing input biomass, as expected given that samples with a lower biomass are more susceptible to environmental contamination. This is seen in FIG. 1D. It was discovered that despite a ˜30% lower biomass at the beginning of library preparation for the SIFT-seq samples, far fewer C. acnes reads were present after SIFT-seq filtering (4223.8 and 119.5 MPM in the highest biomass samples, 1.48 and 0 MPM in the lowest biomass samples, before and after SIFT-seq filtering respectively; FIG. 1D).

Second, SIFT-seq was performed on sheared ΦX174 DNA samples with variable biomass (0.0025-155 ng; FIG. 1E) which was spiked after SIFT-seq tagging with 1 ng of sheared DNA from a well-characterized community of microbes to simulate microbial DNA contamination (10 species; Zymo Research, #D6305). Before applying the SIFT-seq bioinformatics filter, a negative correlation was observed between the ΦX174 DNA input biomass and the relative number of reads from the spike-in community, as expected (Pearson's R=−0.54, p-value=6.5×10−6; Spearman's p=−0.82, p-value=6.3×10−16; FIG. 1E). an average percent decrease of 99.8% of molecules mapping to species of the spike-in community was observed after applying the SIFT-seq filter. This can be seen in FIG. 1F. Sequences mapping to Escherichia coli (E. coli) were the most abundant after filtering (58.89%). Given that ΦX174 genomic DNA is isolated after phage propagation in E. coli culture, it was reasoned that these remaining reads were likely intrinsic to the original sample. Together, these experiments demonstrate the effectiveness of SIFT-seq for the detection and removal of DNA contaminants without removing species originally present in the sample.

### Example 2. Application of SIFT-Seq to Cell-Free DNA in Blood and Urine

Cell-free DNA (cfDNA) in blood and urine has emerged as a useful analyte for the diagnosis of infection. Metagenomic cfDNA sequencing can identify a broad range of potential pathogens with high sensitivity. Yet, because of the low biomass of microbial-derived cfDNA in blood and urine, metagenomic cfDNA sequencing is highly influenced by environmental contamination, limiting the specificity of metagenomic cfDNA sequencing for pathogen identification.

To assess the performance of SIFT-seq in metagenomic cfDNA sequencing, a total of 196 cfDNA samples (154 plasma, 42 urine) collected from six groups of subjects were assayed. The first group was 30 plasma samples from a cohort of 14 patients hospitalized with COVID-19 (“COVID19 cohort”). The second group was 53 plasma samples from a cohort of 44 patients seeking treatment for IBD (4 patients without IBD, 19 patients with Crohn's disease, 21 patients with ulcerative colitis; “IBD cohort”). The third group was 56 plasma samples from a cohort of 44 patients presenting with respiratory symptoms at outpatient clinics in Uganda (“Uganda cohort”). The fourth group was 15 plasma samples from a cohort of 15 patients (10 patients with sepsis, 5 patients without sepsis but in the ICU; “sepsis cohort”). The fifth group was 26 urine samples from a cohort of kidney transplant patients with and without urine culture-confirmed UTIs (16 positive urine culture, 10 negative urine culture; “kidney transplant cohort”). The sixth group was 16 urine samples collected early after transplantation from 10 kidney transplant patients that received a ureteral stent at the time of transplantation (samples were collected pre-stent and post-stent removal for 5 of the 10 patients; “early post-transplant cohort.”

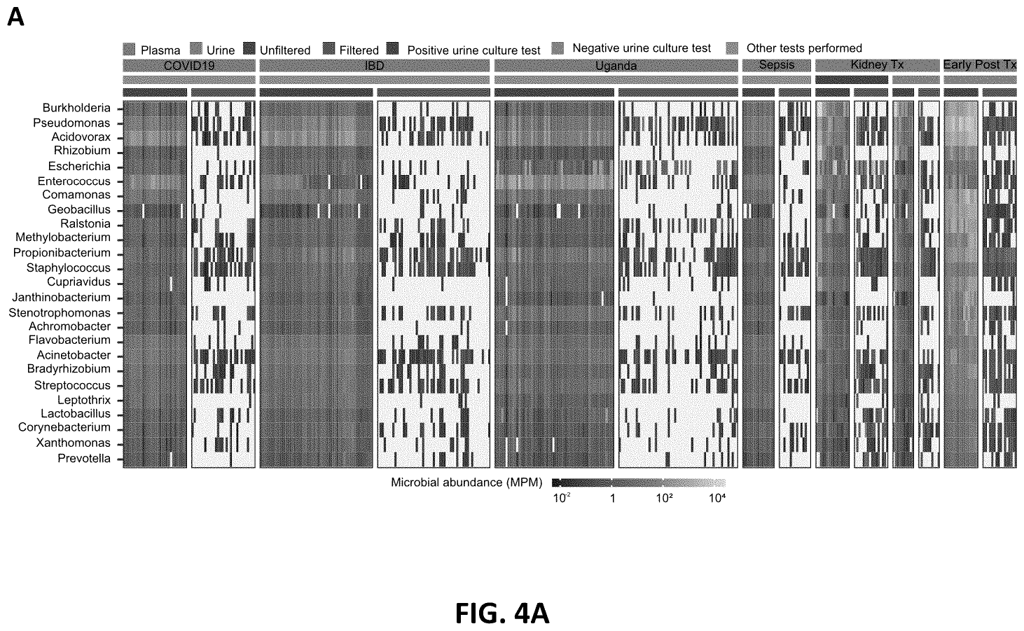

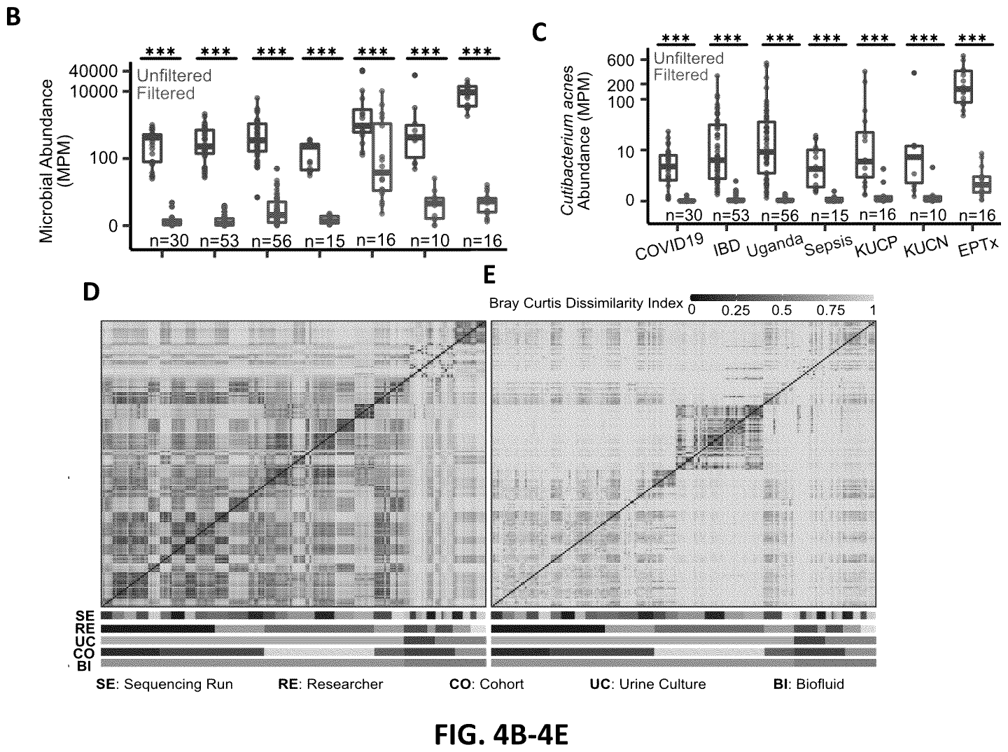

SIFT-seq was performed for all samples and obtained an average of 48.5±23.4 million paired-end reads per sample. The abundance of 68 genera that have been reported as frequent DNA contaminants in multiple independent studies were detected and quantified. FIG. 4A shows the microbial abundance and shows that 49 of these genera were detected in at least one sample. 77% of these genera were completely removed from all samples after SIFT-seq filtering. The total number of molecules from all contaminant genera were calculated and observed an up to 3 orders of magnitude reduction after SIFT-seq filtering (reduced by a factor of 7.5, 1, 711.2, 177.6, 608.8, 215.4, 547.2; two tailed, Wilcoxon signed-rank test, p-values<0.001 for all cohorts; FIG. 4B). The impact of SIFT-seq filtering on removing reads originating from the skin contaminant C. acnes was examined and shown in FIG. 4C. C. acnes was detected in all samples and completely removed from 62 samples by SIFT-seq filtering. An up to 2 orders of magnitude reduction of C. acnes reads was observed in the remaining samples (two tailed, Wilcoxon signed-rank test, p-values<0.001 for all cohorts).

Next, SIFT-seq was evaluated for its utility to correct for batch effects and to reveal true differences in microbiome profiles for different patient groups. To this end, the Bray-Curtis Dissimilarity Index was calculated for all clinical samples included in this study and sorted the datasets based on the following parameters: 1) sequencing run, 2) operator, 3) urine culture test, 4) study cohort, and 5) biofluid type. Before SIFT-seq filtering, a high similarity for samples assayed in the same experimental batches was observed as shown in FIG. 4D. SIFT-seq filtering removed these batch effects and revealed distinct cohort-specific microbiome profiles. Most notably, distinct plasma microbiome profiles for plasma samples from the Uganda cohort were observed, FIG. 4E. These results demonstrate that SIFT-seq directly applied to biofluids leads to a dramatic decrease in experimental noise and bias due to DNA contamination.

### Example 3. SIFT-Seq Enables to Screen for UTI and to Characterize the Urine Microbiome

The healthy urinary tract was long believed to be sterile, but this picture was challenged with recent advances in urine culture techniques that have identified bacteria in the urinary tract of both males and females. Gottschick, C. et al. Microbiome 5, 99 (2017). Yet many microbes are difficult to cultivate in vitro, and bacterial culture can also be sensitive to contamination. Therefore, comprehensive and accurate characterization of species colonizing the urinary microbiome is still lacking.

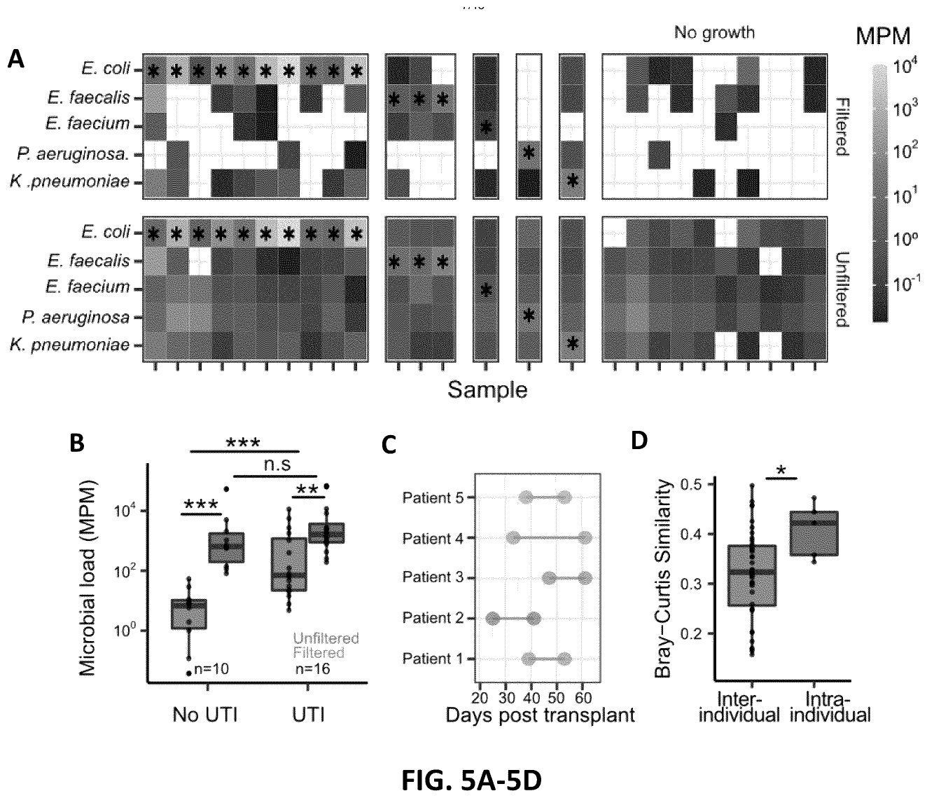

SIFT-seq could provide insight into the composition of the urine microbiome with both high sensitivity and specificity. SIFT-seq was first applied to 26 urine samples from 23 kidney transplant patients with and without infection of the urinary tract as determined by conventional urine culture (16 positive urine culture [Enterococcus faecalis: n=3; Enterococcus faecium: n=1; Escherichia coli: n=10; Klebsiella pneumoniae: n=1; Pseudomonas aeruginosa: n=1] and 10 negative urine culture). SIFT-seq consistently identified microbial cfDNA from species reported by urine culture (16/16 urine culture positive samples; FIG. 5A). SIFT-seq also identified two Corynebacterium species (Corynebacterium jeikeium and Corynebaterium urealyticum) in one sample from a urine culture positive patient (E. coli) with culture confirmed Corynebacterium co-infection. In addition, samples from positive urine culture patients had a significantly higher burden of total microbial DNA compared to samples from negative urine culture patients (1451.8±3024.7 MPM and 12.8±17.6 MPM, respectively in the filtered samples; p-value=7.1×10−4, two tailed, Wilcoxon rank-sum test, FIG. 5B). Conventional metagenomic sequencing (without SIFT-seq filtering) detected uropathogens with equal sensitivity but was not robust against environmental contamination: DNA from common uropathogens not identified by culture was detected in many samples, albeit with low abundance, including in samples from patients without urine culture-confirmed UTIs. Therefore, the improved specificity of SIFT-seq allows for more accurate characterization of co-infection networks in the scope of UTIs, and more accurate characterization of the normal urine microbiome in the absence of UTIs. It is important to note that two common skin microbes, C. acnes and Staphylococcus epidermidis, were found in most samples (23/26 samples). While these two species have been shown to cause UTIs, they may also have been introduced as contaminants at the time of urine collection, which underscores an important limitation of SIFT-seq: SIFT-seq is not robust against contamination that occurs before the tagging step.

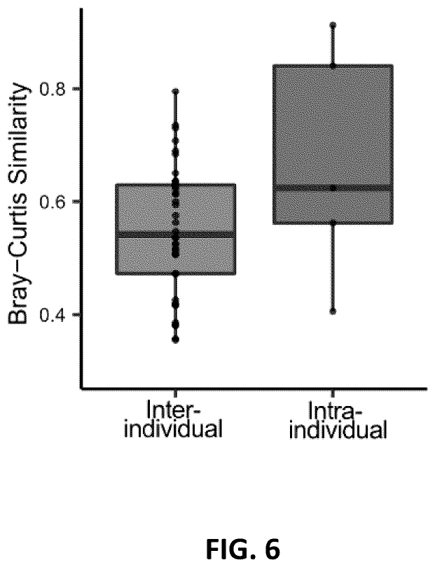

Studies investigating the temporal dynamics of urine microbiome in individuals can benefit from the high sensitivity and specificity achieved with our assay. SIFT-seq was applied to paired urine samples obtained from 5 kidney transplant patients collected at two time points before and after ureteral stent removal (FIG. 5C). The similarity of microbial composition was compared between samples from the same patient (intra-individual) and between different patients (inter-individual) at different sampling points. Using filtered but not the unfiltered datasets, it was observed that the microbial composition remained more similar in the same patient than between different patients, as is seen in FIG. 5D. These results support the utility of SIFT-seq to measure subtle dynamics in urine microbiome composition (Mean Bray-Curtis Similarity: 0.41±0.06 and 0.317±0.09 respectively, p-value=2.8×10−2, two tailed, Wilcoxon rank-sum test, FIG. 6).

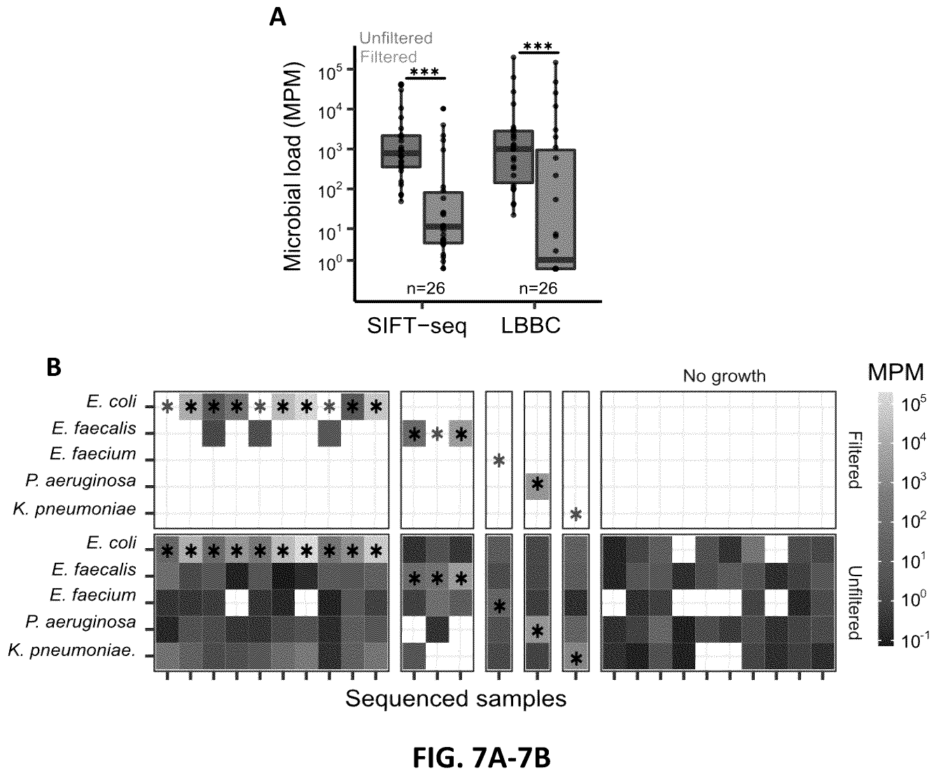

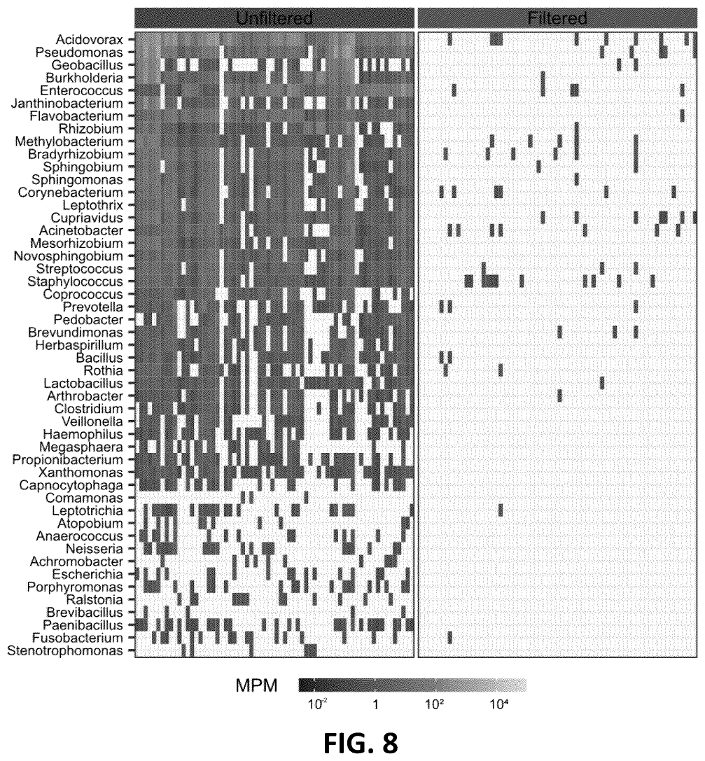

To evaluate the performance of SIFT-seq to existing bioinformatic techniques for eliminating environmental DNA contamination, SIFT-seq was benchmarked against Low Biomass Background Correction (LBBC), a bioinformatics noise filtering tool for eliminating environmental DNA contamination. LBBC identifies and removes two types of noise, 1) digital cross talk stemming from alignment errors, and 2) physical noise arising from environmental DNA contamination present in reagents required for DNA isolation and sequencing libraries preparation. SIFT-seq-filtered and LBBC-filtered data were compared for samples from the kidney transplant cohort (n=26). On average, LBBC filtering resulted in a 1.4-fold reduction of reads originating from contaminant genera, while SIFT-seq achieved a 7.5-fold reduction (p-valueSIFT-seq <0.001, p-valueLBBC<0.001, two-tailed, Wilcoxon signed-rank test) (FIG. 7A). SIFT-seq identified all species detected from conventional urine culture (16/16) while LBBC only detected 10/16 species reported by culture (FIG. 7B). The decrease in false positive rate after LBBC filtering occurred at the expense of decreased true positive rate. SIFT-seq was also performed on negative controls included in 32/33 experimental batches. the reads originating from contaminant genera were quantified before and after SIFT-seq filtering and it was found that SIFT-seq removed 95.8% of all contaminant genera detected in the negative controls (506.7±827.53 versus 0.4±0.6 MPM before and after SIFT-seq filtering, respectively (p-value<0.001, two-tailed, Wilcoxon signed-rank test) (FIG. 8).

### Example 4. SIFT-Seq Identifies Bacterial and Viral Co-Infection of COVID-19 from Blood

The COVID-19 pandemic is an unprecedented human health crisis. Viral or bacterial co-infection occurs in roughly 4% of hospitalized COVID-19 patients but can occur in up to 30% of COVID-19 patients admitted to the intensive care unit. Co-infection has been associated with longer fever duration, and increased risk of intensive care unit admission and need for mechanical ventilation. SIFT-seq may offer sensitive detection of bacterial and viral co-infection in COVID-19 patients with improved specificity over conventional metagenomic sequencing assays.

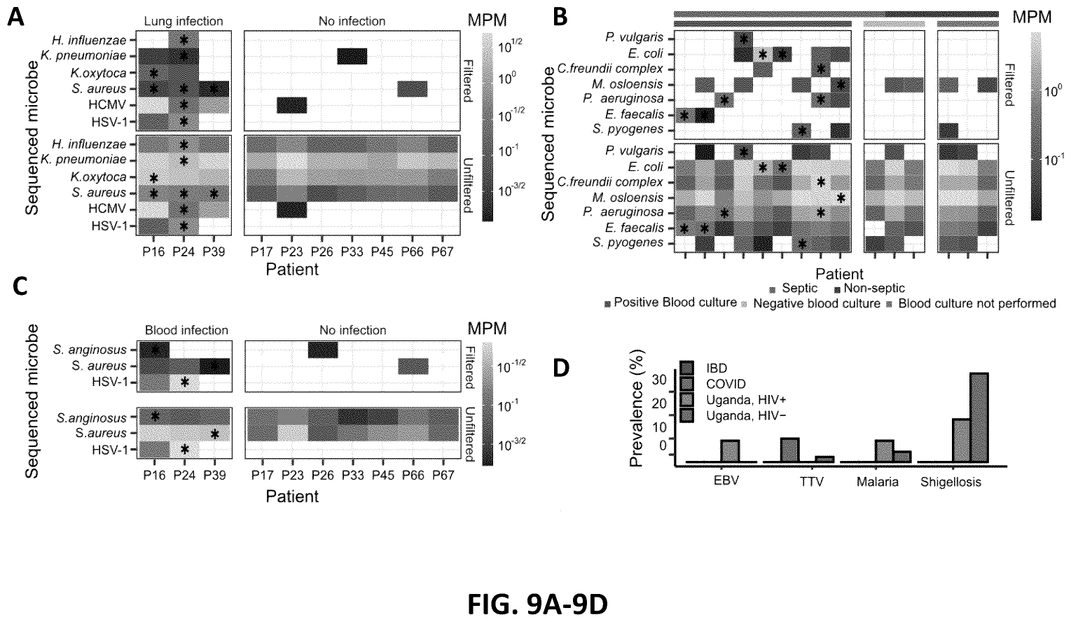

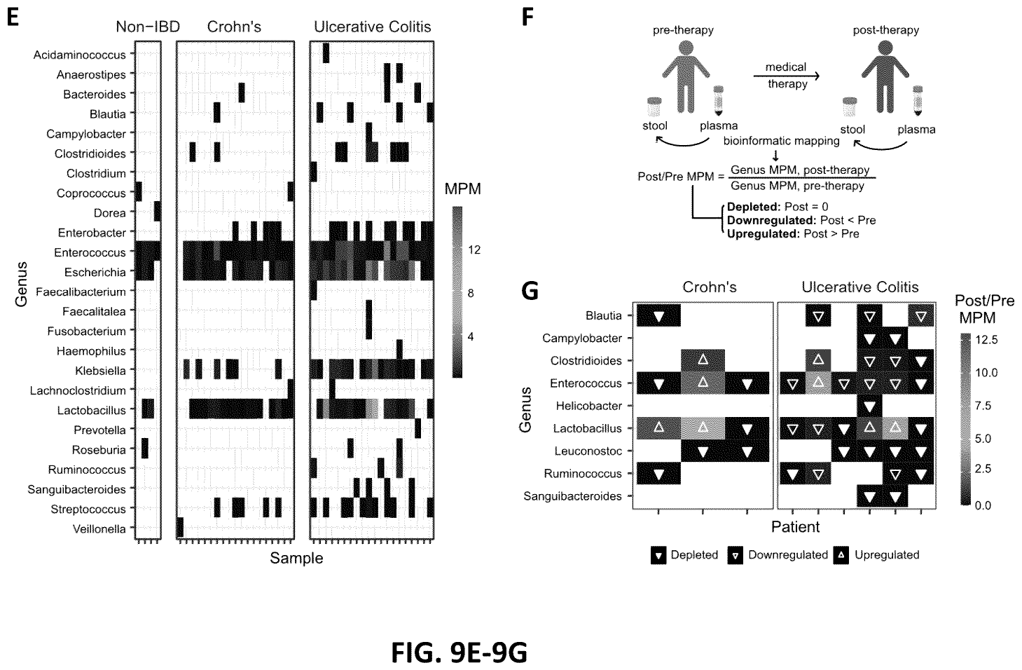

SIFT-seq was applied to 30 plasma samples from 14 patients with COVID-19 collected as part of a clinical study aimed at identifying predictors of disease severity. Respiratory and blood cultures were obtained as part of standard clinical care. Three patients (P16, P24, P39) tested positive for bloodstream infection and respiratory tract infection, while all other patients were not diagnosed with COVID-19 co-infection. SIFT-seq identified the causative pathogen in 3/3 bloodstream infection cases and 8/8 respiratory infection cases (FIGS. 9A and B). Conventional metagenomic sequencing (without SIFT-seq filtering) was equally sensitive to these pathogens but was limited by specificity. Of interest, while plasma collected the day of infection for P24 was not obtained, cfDNA was identified originating from K. pneumoniae and Haemophilus influenzae, for which the patient tested positive four days later. These results suggest that SIFT-seq may be able to identify cases of infection earlier than traditional culture methods, and with improved specificity compared to conventional metagenomic sequencing techniques.

### Example 5. SIFT-Seq Identifies Infection Causing Pathogens in Sepsis Patients

Sepsis is a life-threatening organ dysfunction caused by dysregulated host response to a bacterial, viral, fungal, or parasitic infection. According to the World Health Organization, in 2017 there were 48.9 million sepsis cases and 11 million sepsis-related deaths worldwide. When sepsis is suspected, broad-spectrum empiric antibiotics are administered, and tests are performed to identify the infection-causing pathogens. Blood culture is the gold standard method to detect infectious pathogens in the bloodstream, however this method is time consuming and limited to few culturable microbes. Though other molecular tests can shorten time to results when performed directly on blood, the low microbial burden in blood leads to low sensitivity, low negative predictive values, and detection of only a few specific pathogens. Chun, K. et al. J. Lab. Autom. 20, 539-561 (2015). Thus, conventional metagenomic cfDNA sequencing holds promise in identifying sepsis-causing pathogens.

The utility of SIFT-seq to identify sepsis-causing pathogens in patients with sepsis was tested. For this, a blinded analysis was performed on 15 plasma samples (n=10 from septic patients and n=5 from non-septic patients. 9/15 patients had a positive blood culture result (9/10 patients with sepsis), 3/15 had negative blood culture and for 3/15 patients, blood culture was not performed. A total of 10 pathogens were identified in the 9 blood-culture positive samples. After unblinding a strong agreement was found between pathogens that were identified by blood culture and those that were identified by SIFT-seq: SIFT-seq detected 10 out of 10 of pathogens reported by blood culture. Importantly, for only 2/9 patients with positive blood culture, a plasma sample was collected at the time of the positive blood culture (E. faecalis identified by blood culture and SIFT-seq, FIG. 9C). For 7/9 patients, the plasma sample for SIFT-seq was collected after the initial positive blood culture and after initiation of antibiotic treatment. Blood cultures corresponding to the time of plasma sample collection for those 7/9 samples were all negative, while SIFT-seq correctly identified the pathogen identified by culture in the sample before initiation of antibiotic treatment. This experiment demonstrates the utility of SIFT-seq to identify blood-borne pathogens in the setting of sepsis, even after initiation of antibiotic treatment when blood cultures frequently fail.

### Example 6. SIFT-Seq Identifies Clinically Relevant Bacterial and Viral Microorganisms with Low Prevalence and Low Microbial Burden

Neglected tropical diseases significantly impact the public health and economies of low-income countries. Treatments exist for many of these diseases, but development and deployment of reliable diagnostic tests has been slow. SIFT-seq could be used to screen for infections with low prevalence and low microbial burden.

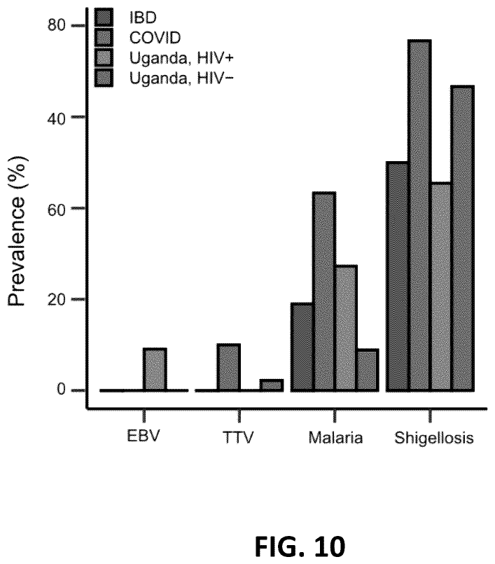

SIFT-seq was applied to 56 plasma samples from 44 individuals who presented with symptoms of respiratory illness at outpatient clinics in Uganda. Nine of these individuals were HIV positive at the time of sample collection. The data was mined to determine the prevalence of clinically-relevant bacterial and viral microorganisms endemic to Uganda and compared with results obtained for plasma samples collected from subjects that live in North America (53 plasma samples from the IBD cohort; 30 plasma samples from the COVID-19 cohort). The samples were screened for Epstein-Barr virus, Torque Teno virus, and pathogens associated with malaria (Plasmodium vivax and P. falciparum), and shigellosis (Shigella sonnei, S. dysenteriae, S. boydii, and S. flexneri) before (FIG. 10) and after SIFT-seq filtering (FIG. 9D). After SIFT-seq filtering, these microorganisms were found at varying rates in samples from the Uganda cohort: malaria (3/44), Epstein-Barr virus (1/44), shigellosis (19/44), and torque teno virus (1/44), but not in the IBD cohort. Torque teno virus, which has previously been reported to be elevated in immunocompromised patients, was identified in 3/30 COVID-19 patient samples, all from patients who had received a bone marrow transplant prior to sample acquisition.

### Example 7. SIFT-Seq Identifies Signatures of Bacterial Translocation from the Gastrointestinal Tract

Bacterial translocation of intestinal microbes through mucosal membranes is believed to be a normal phenomenon but has been found to occur more frequently in patients experiencing gut flora disruption. In patients with inflammatory bowel disease, gut vascular barrier disruption has been linked to increased intestinal permeability and subsequent microbial translocation across the mucosal membrane. The translocation of gut bacteria and their products to extraintestinal sites can result in systemic inflammation, resulting in autoimmune or other non-infectious diseases. Detecting signatures of translocation is therefore important but difficult in view of the low abundance of microbial DNA due to translocation in blood.

To identify signatures of bacterial translocation, whole genome shotgun sequencing of fecal samples from 44 patients (non-IBD n=4, Crohn's n=19, ulcerative colitis, n=21) were compared to matched plasma cfDNA samples assayed using SIFT-seq. Bacterial species identified in matched fecal and plasma samples were quantified as shown in FIG. 9E. cfDNA derived from gut-specific microbes was identified in all patient samples, though to a much greater extent in individuals with ulcerative colitis (0.57±0.65, 1.22±1.38, and 5.55±9.46 MPM of gut-specific bacteria for non-IBD, Crohn's disease, and ulcerative colitis samples, respectively). To investigate the effects of treatment on bacterial translocation, additional stool and plasma samples were collected from nine patients (Crohn's n=3, ulcerative colitis n=6) after treatment initiation and performed whole genome shotgun sequencing of stool and SIFT-seq on plasma cfDNA, seen in FIG. 9F. The relative abundance of gut-specific bacterial species was quantified before and after treatment and found that the burden of cfDNA decreased for most bacterial species (28/36) following treatment, which may be explained by a reduction in the degree of bacterial translocation with treatment (FIG. 9G). Of interest, out of seven subjects for which Lactobacillus was detected before treatment, five displayed an increase in Lactobacillus species burden in blood after treatment (up to 12.7-fold increase after treatment and an average of 3.36-fold MPM increase after treatment across all samples). Lactobacillus has been shown to promote gastrointestinal barrier function, protecting the gut from pathogenic bacteria and preventing inflammation. For bacterial species besides Lactobacillus, an average of 70% reduction in MPM after treatment is found. These preliminary results support the use of SIFT-seq to identify subtle signatures of bacterial translocation in the blood.

### General Methods Used in Examples.

### Study Cohorts and Sample Collection:

### Uganda Cohort and Sample Collection

Forty-four plasma samples were collected from individuals that presented with respiratory symptoms at outpatient clinics in Uganda. Briefly, peripheral blood was collected in Streck Cell-Free BCT (Streck #230257) and centrifuged at 1600×g for 10 minutes. Plasma was stored in 1 mL aliquots at −80° C. The study was approved by the Makerere School of Medicine Research and Ethics Committee (protocol 2017-020). All patients provided written informed consent.

### IBD Cohort Sample Collection

Peripheral blood samples were collected under IRB approved protocol (1806019340) at the Jill Roberts Center for IBD at Weill Cornell Medicine. PBMCs and plasma were fractionated using a Ficoll-Hypaque gradient. Informed consent was obtained from all participants

### IBD Cohort Fecal Sample Collection

DNA from fecal samples was isolated using the MagAttract PowerMicrobiome DNA/RNA kit with glass beads (Qiagen, Germany). Metagenomic libraries were prepared using the NEBNext Ultra II for DNA Library Prep kit (New England Biolabs, Ipswich, MA) following the manufacturer's protocol. The DNA library was sequenced on an Illumina HiSeq instrument using a 2×150 paired-end configuration in a high output run mode. Informed consent was obtained from all participants

### COVID-19 Cohort Sample Collection

Peripheral blood samples were collected as part of an observational study among individuals with COVID-19 that were treated at New York-Presbyterian/Weill Cornell Medical Center (NYP-WCMC) and Lower Manhattan Hospital under IRB approved protocol (IRB 20-03021645). Informed consent was obtained from all participants. PBMCs and plasma were fractionated using a Ficoll-Hypaque gradient.

### Sepsis Cohort Sample Collection

Since 2014, investigators have prospectively consented patients admitted to any ICU at NYP-WCMC to participate in a registry involving collection of biospecimens and clinical data. For each participant, whole blood (6-10 mL) was obtained. Whole blood samples were drawn into EDTA-coated blood collection tubes (BD Pharmingen, San Jose, CA). Samples were stored at 4° C. and centrifuged within 4 hours of collection. Plasma was separated and divided into aliquots and kept at −80° C. The registry was approved by the institutional review board of WCMC (1405015116, 20-05022072).

### Kidney Transplant Cohort Sample Collection

Twenty six urine samples were collected from 23 kidney transplant recipients who received care at NYP-WCMC. The study was approved by the Weill Cornell Medicine Institutional Review Board (protocol 1207012730). All patients provided written informed consent. Patients provided urine specimens using a clean-catch midstream collection protocol. The urine specimen was centrifuged at 3000×g for 30 minutes and supernatant was stored as 1 mL of 4 mL aliquots.

### Early Post Transplant Sample Collection

Urine specimens collected within 10±5 days of ureteral stent removal from patients who agreed to participate in the WCM IRB approved protocol #20-01021269 were included in this study. Urine specimens were collected within 47±11 days post-kidney transplantation. The presence of UTI was excluded by a negative urine culture and the absence of pyuria. This study was approved by the Weill Cornell Medicine Institutional Review Board (protocol 20-01021269).

### Definition of Positive and Negative Urine Culture for the UTI and Early Post-Transplant Cohorts

A positive urine culture was defined as a culture growing an organism identified to at least the genus level (≥10,000 cfu/mL). A urine culture was defined as negative when either no organism was isolated in culture (<1000 cfu/mL) or the organism was unidentified to either the genus or species level (i.e., unidentified) and the colony count was <10,000 cfu/mL.

SIFT-seq in plasma. An aliquot of 520 μL of plasma is centrifuged at 15,000 RPM for 5 minutes to pellet cellular debris. The supernatant is transferred to a new 1.5 mL tube and 500 μL of PBS is added, and the solution is heated to 98° C. for 10 minutes and mixed at 1000 RPM to coagulate the albumin present in plasma. The solution is then centrifuged at 7500 RPM for 10 minutes. 500 μL of supernatant is transferred to 3.25 mL of ammonium bisulfite solution (Zymo Research, product #5030) and heated to 98° C. for 10 minutes. Samples are then kept at 54° C. for 60 minutes. Then, cfDNA extraction is performed using commercially available column-based kits (Qiagen, product #55114). Prior to DNA elution, DNA desulphonation buffer (Zymo Research, product #5030) is added to the columns for 20 minutes, followed by two washes with 200 proof ethanol. DNA is then eluted according to manufacturer recommendations, and single-stranded library preparation is performed (Claret Biosciences, product #CBS-K150B). Libraries are then sequenced on an Illumina sequencer.

SIFT-seq in urine. An aliquot of 520 μL of plasma is centrifuged at 15,000 RPM for 5 minutes to pellet cellular debris. 500 μL of supernatant is transferred to 3.25 mL of ammonium bisulfite solution (Zymo Research, product #5030) and heated to 98° C. for 10 minutes. Samples are then kept at 54° C. for 60 minutes. Then, cfDNA extraction is performed using commercially available column-based kits (Norgen Biotek, product #56700). Prior to DNA elution, DNA desulphonation buffer (Zymo Research, product #5030) is added to the columns for 20 minutes, followed by two washes with 200 proof ethanol. DNA is then eluted according to manufacturer recommendations, and single-stranded library preparation is performed (Claret Biosciences, product #CBS-K150B). Libraries are then sequenced on an Illumina sequencer.

Sequencing Library Preparation. Bisulfite conversion of cfDNA involves a cfDNA denaturing step at 98° C. such that we get single stranded cfDNA molecules after DNA extraction. For this reason, a single stranded sequencing library preparation method is chosen for the next steps. We prepared sequencing libraries using the SRSLY PicoPlus DNA NGS Library Preparation Base Kit (SRSLY Cat #CBS-K250B-24) with the SRSLY UDI Primer Set-24 (SRSLY Cat #CBS-UD-24) following the manufacturer's protocol, with the following modifications:

The input cfDNA volume used was 18 μL.
1.25 μL of NGS Adapters A and 1.25 μL of NGS Adapters B were added to the 20 μL denatured DNA reaction tube, and the volume was completed by 1.5 μL of ultrapure water.
The Index PCR Master Mix was substituted for an equal volume of KAPA HiFi Uracil+Ready Mix (2×).
The Indexed Library DNA Purification step was performed twice, first eluting in 50 μL and then in 25 μL.

Alignment to the human genome. Adapter and low-quality bases from the reads were trimmed using BBDuk (BBDuk V38.4634, --entropy=‘0.25’ --maq=‘10’ -Xmx1g tbo tpe) and aligned to the C-to-T and G-to-A converted human genome using Bismark (Bismark-0.22.135, --unmapped, --quiet). PCR duplicates were removed using Bismark.

Depth of coverage. The depth of sequencing was measured by summing the depth of coverage for each mapped base pair on the human genome after duplicate removal and dividing by the total length of the human genome (hg19, without unknown bases).

Removing unconverted molecules. Aligned BAM files are filtered to remove unconverted molecules using the Bismark (Bismark-0.22.1) alignment package with default parameters.

Bisulfite conversion efficiency. Bisulfite conversion efficiency was estimated by quantifying the rate of C [A/T/C] methylation in human-aligned reads (using MethPipe V3.4.336), which are rarely methylated in mammalian genomes.

Pre-processing of the unmapped reads. Reads originating from the Phix genome were removed from the host unmapped reads using Bowtie 237 (Bowtie 2.4.3, --local, --very-sensitive-local, --un-conc). Read IDs from the remaining reads were used to subset paired end reads from the original FASTQ files. Adapter trimming and read quality filtering was performed using BBDuk (BBDuk V38.46, maq=32). Remaining reads were deduplicated using samtools38 (samtools V1.14) and merged using FLASH239 (-q -M75 -O). K-mer decontamination to remove human reads was then performed using BBDuk (BBDuk V38.46, k=50, prealloc=t) and the obtained fastq file was converted to a fasta file for metagenomics analysis.

Metagenomic abundance estimation from sequencing data. Reads mapping to microbial species were identified using HS-BLASTN⁴⁰ (hs-blastn-1.0.0) and microbial abundances were estimated using GRAMMy (version 1)⁴¹. Specific to SIFT-seq, read-level filtering of contaminants is performed by removing sequenced reads with 4 or more cytosines present, or one methylated CpG dinucleotide (the latter represents unmapped, human-derived molecules). Species-level filtering based on the distribution of mapped reads is carried out by first aligning filtered and unfiltered datasets independently. Cytosine-densities of mapping-coordinates in both datasets are measured using custom scripts, and their distributions are compared using a Kolmogorov-Smirnov test. Significantly different filtered-unfiltered distributions are further processed (D-statistic>0.1 and p-value<0.01). Briefly, filtered datasets whose distribution of cytosines at mapped locations is significantly lower than unfiltered datasets have one read removed, and are re-tested for differences in their distribution. If the distributions are more similar (as measured through the same criteria), it is filtered out. This process is repeated until distributions are no longer significantly different, or if all reads are removed. Read and species level filtering was performed using custom scripts written in Python. Microbial abundance in downstream analyses was quantified as Molecules Per Million reads (MPM).

$$M P M = \frac{Adjusted Blast hits \times {10}^{6}}{Total Trimmed Reads} \qquad ( 1 )$$

Benchmarking SIFT-seq against Low Background Biomass Correction (LBBC). To benchmark SIFT-seq, SIFT-seq performance was compared to Low Microbial Biomass Background Correction (LBBC) tool. For this, datasets from the UTI cohort were used from this study and matched to standard sequenced datasets from a previously published study. Default LBBC filtering parameters were used for this analysis (ΔCVmax=2, 82 min=−5.5).

Identification of translocated gut bacteria in plasma. Fecal shotgun metagenomic data for 53 samples was obtained from 44 patients diagnosed with inflammatory bowel disease (IBD). Low-quality bases and Nextera-specific sequences were trimmed using Trim Galore V 0.6.5 (Trim Galore V 0.6.5, --nextera --paired). Reads were aligned against the human references (UCSC hg19) using Bowtie2 (Bowtie 2.4.3, --maxins 700 --no-discordant --score-min L, 0, −0.2). Unaligned reads were extracted and assembled with metaSPAdes42 (SPAdes 3.15.3; --meta) and classified with Kaiju (Kaiju 1.7.4). Paired cfDNA samples were filtered with SIFT-seq pipeline and aligned to the assembled reads with Bismark (Bismark 0.22.1). Mapped reads with a minimum quality score of 15 were extracted and filtered for gut-specific microorganisms identified by The Human Gut Microbiome Atlas.

Statistics and reproducibility. All statistical methods were performed in R version 4.0.5. Groups were compared using a two-sided Wilcoxon Signed Rank or Wilcoxon Rank Sum tests. Boxes in the boxplots indicates 25th and 75th percentile, the band in the box indicated the median and whiskers extend to 1.5× Interquartile Range (IQR) of the hinge. As many patient samples were collected as possible that fit the criteria in each cohort. Data from 3 samples that were collected by Foley from the Kidney Transplant cohort samples were excluded, included samples were all collected by clean-catch method; also, data from 4 samples that had mixed urine culture results or associated with positive urine culture from the Early Post Transplant cohort were excluded, included samples were all urine culture negative. Investigators were blinded to group allocation during data collection of samples in the Sepsis cohort. Groups, and detailed clinical information (e.g. data from conventional blood cultures) were shared with the investigators after the data was analyzed and shared with collaborators who then shared metadata elements. For the other groups, blinding was not implemented the study was focused on the development of a new method and because in the case of the Kidney Transplant and Uganda cohorts group allocations were available from prior studies by the same investigators. Experiments were not randomized.

What is claimed is:

1. A method of differentiating sample-intrinsic nucleic acids from contaminant nucleic acids, the method comprising:
providing a sample containing nucleic acids;
tagging the nucleic acids in or from the sample;
subjecting the nucleic acids after tagging to nucleic acid sequencing; and
differentiating sample intrinsic nucleic acids from contaminant nucleic acids based on tag and sequence analysis.

2. The method of claim 1, wherein the sample is obtained from a host and the sequence analysis comprises aligning the nucleic acid sequences to a host genome thereby identifying sequences from the host.

3. The method of claim 1 or 2, wherein the tag and sequence analysis comprises:
filtering the nucleic acid sequences into a preliminary group of tagged sequences and a preliminary group of untagged sequences.

4. The method of any one of claims 1-3, wherein the tag and sequence analysis comprises:
aligning the nucleic acid sequences with a database and identifying the contaminant reads.

5. The method of claim 1, wherein the sample is obtained from a host, and the tag and sequence analysis comprises:
aligning the nucleic acid sequences to a host genome thereby identifying sequences from the host, and removing the host sequences;
separating the remaining nucleic acid sequences into a preliminary group of tagged sequences and a preliminary group of untagged sequences;
aligning the preliminary group of tagged sequences and the preliminary group of untagged sequences to a database thereby determining the species origin of the preliminary group of untagged sequences; and
identifying the tagged and untagged sequences of the same species origin as contaminant sequences.

6. The method of any one of claims 1-5, wherein sequences associated with contamination are removed from the reads.

7. The method of any one of claims 1-6, further comprising extracting the nucleic acids from the sample after the tagging and before subjecting the extracted nucleic acids to nucleic acid sequencing.

8. The method of any one of claims 1-5, further comprising extracting the nucleic acids from the sample before the tagging.

9. The method according to claims 1-8, wherein the sample comprises viruses, microorganisms, plants, and/or mammalian cells, and the nucleic acids are extracted therefrom for sequencing.

10. The method according to claim 9, wherein the microorganisms are parasites, protists, archaea, bacteria, and/or fungi.

11. The method according to claim 9, wherein the mammalian cells are human, rodent, primate, equine, canine, bovine, and porcine cells.

12. The method according to claims 1-9, wherein the sample is a biological sample obtained from a mammalian host subject.

13. The method according to claim 12, wherein the biological sample is a cell free sample comprising DNA.

14. The method according to claim 12, wherein the biological sample obtained from mammalian host subject is a nasal swab, a saliva sample, a skin sample, a urine sample, a gastrointestinal tract sample, a tissue sample, a fecal sample, a blood sample, a plasma sample, cerebrospinal fluid sample, peritoneal fluid sample, pleural effusion, and/or a serum sample.

15. The method according to claims 1-12, wherein the sample is an environmental sample.

16. The method according to claim 15, wherein the environmental sample is air, water, soil, biological materials, and wastes, wherein the wastes are liquids, solids or sludges.

17. The method according to any of the preceding claims, wherein the tagging comprises modifying one or more nucleotides of the nucleic acids in the sample.

18. The method of claim 17, wherein the modifying comprises converting cytosine nucleotides into uracil nucleotides.

19. The method of claim 18, wherein the converting cytosine nucleotides into uracil nucleotides is achieved by treating the nucleic acids with a bisulfite salt.

20. The method of claim 18, wherein the converting cytosine nucleotides into uracil nucleotides is achieved by treating the nucleic acids with a cytosine deaminase, a cytidine deaminase (CDA), or a dCMP deaminase (DCTD).

21. The method of claim 20, wherein the CDA is an apolipoprotein B editing complex (APOBEC) and/or an activation-induced deaminase.

22. The method of claim 21, wherein the APOBEC is APOBEC1, APOBEC3A-H, and/or APOBEC3G.

23. The method of claim 17, wherein the modifying comprises converting adenosine nucleotides to inosine nucleotides.

24. The method of claim 23, wherein the converting adenosine nucleotides to inosine nucleotides is achieved by treating the nucleic acids with an adenosine deaminase.

25. The method of claim 24, wherein the adenosine deaminase is AMP deaminase (AMPD1), adenosine deaminase 1 (ADA1), and/or adenosine deaminase 2 (ADA2).

26. The method of claim 17, wherein the modifying comprises converting guanosine nucleotides to xanthosine nucleotides.

27. The method of claim 26, wherein the converting guanosine nucleotides to xanthosine nucleotides is achieved by treating the nucleic acids with a guanine deaminase (GDA).

28. The method according to claim 18 or 19, further comprising after the nucleic acid sequencing:
identifying nucleic acids that do not have cytosines replaced with uracils as contaminant nucleic acids;
identifying nucleic acids that have cytosines replaced with uracils as intrinsic nucleic acids; and/or
identifying nucleic acids that have cytosines replaced with uracils wherein the uracils are converted to thymines in subsequent DNA synthesis steps as intrinsic nucleic acids.

29. The method of any one of claims 1-8, wherein the tagging comprises attaching a compound to the nucleic acids.

30. The method of claim 29, wherein the attaching is by way of a covalent linkage.

31. The method of claim 29, wherein the compound is selected from biotin, a peptide, an alkyne, an azide, a BrdU, an IrdU, an amine, or an oligonucleotide.

32. The method of claim 31, wherein the peptide is a HIS tag.

33. The method of claim 31, wherein the oligonucleotide is a primer sequence.

34. The method of either claim 29 or 31, further comprising isolating the nucleic acids attached with the compound before subjecting the isolated nucleic acids to nucleic acid sequencing.

35. The method according to any of the preceding claims, wherein the extracting the nucleic acids comprises lysing the cells in the sample to release the nucleic acids.

36. The method according to any of the preceding claims, wherein the sample is suspected to contain a pathogenic microorganism.

37. The method according to any of the preceding claims, wherein the nucleic acid sequencing is metagenomic sequencing.

38. The method according to any of the preceding claims, further comprising enriching the non-contaminant nucleic acids by their tags.
